(19) United States
(12) Patent Application Publication
(10) Pub. No.: US 2026/0315184 A1
(43) Pub. Date: Oct. 15, 2026

(54) REPROCESSOR, METHOD, AND COMPUTER-READABLE MEDIUM FOR CONTROLLING SUPPLY OF FLUID INTO AN INTERNAL CONDUIT OF MEDICAL EQUIPMENT INCLUDING AN ENDOSCOPE

(71) Applicant: OLYMPUS MEDICAL SYSTEMS CORP., Tokyo (JP)

(72) Inventor: Kohei SEKINO, Tokyo (JP)

(73) Assignee: OLYMPUS MEDICAL SYSTEMS CORP., Tokyo (JP)

(21) Appl. No.: 19/468,026

(22) Filed: Feb. 3, 2026

Related U.S. Application Data
(63) Continuation of application No. PCT/JP2023/029255, filed on Aug. 10, 2023.

(51) Int. Cl. A61B 90/70; A61B 1/12

(52) U.S. Cl. CPC A61B 90/70; A61B 1/123; A61B 1/125; A61B 2090/701

(57) ABSTRACT

A reprocessor includes a first tube connectable to a first conduit of medical equipment with a gap therebetween, a second tube connectable to a second conduit of the medical equipment, a supplying apparatus configured to supply a fluid to the first and second conduits via the respective tubes, and a processor. The processor controls the supplying apparatus to start supplying the fluid to the second conduit at a first timing, and to start supplying the fluid to the first conduit at a second timing before the fluid supplied to the second conduit flows out from the gap via a merging point and the first conduit, thereby pushing the fluid back toward the merging point. The medical equipment includes an internal conduit including the first conduit, the second conduit, and a third conduit, which are open at one end and merge with each other at the merging point at another end.

### CROSS REFERENCE TO RELATED APPLICATION

This application is a continuation of International Application No. PCT/JP 2023/029255, filed on Aug. 10, 2023, the entire contents of which are incorporated herein by reference.

### FIELD

The present disclosure relates to a reprocessor, a method, and a non-transitory computer-readable medium for controlling supply of fluid into an internal conduit of medical equipment including an endoscope.

### BACKGROUND

Conventionally, endoscopes as medical equipment have been widely used. Endoscopes are configured to observe an inside of a subject by an insertion portion being inserted into the subject when in use. In addition, endoscopes, when in use, are configured to perform treatment with a treatment instrument inserted through a treatment instrument channel (internal conduit) as needed, and perform suction from the inside of a subject, via a suction channel (internal conduit). After the endoscopes are used, the endoscopes are reprocessed in preparation for the next use.

In reprocessing, not only cleaning, disinfecting, sterilizing, and the like of a surface of an endoscope, but also cleaning, disinfecting, sterilizing, and the like of an internal conduit of the endoscope are performed.

The cleaning, disinfecting, sterilizing, and the like of the internal conduit of the endoscope are performed by connecting a connector of a cleaning tube to a ferrule provided in an opening of the internal conduit and by pouring a cleaning solution into the internal conduit of the endoscope through the cleaning tube. At this time, if the connector of the cleaning tube is connected to the ferrule so as to block the ferrule, the cleaning solution or the like is not allowed to reach a connecting part, and the ferrule cannot be cleaned sufficiently. As a result, the ferrule is required to be cleaned separately.

In view of the above, for example, Japanese Patent No. 6010273 discloses an endoscope reprocessor configured to eliminate a need for cleaning a ferrule separately by using a connector which has a structure in which a gap is created when a connector of a cleaning tube is connected to the ferrule of an internal conduit of an endoscope.

The above-described Japanese Patent No. 6010273 describes that connectors of cleaning tubes A and B are connected respectively to ferrules of two openings of the internal conduit of the endoscope, which is branched into three, and perform the following three kinds of control (1) to (3), thereby suppressing a clogging of the gap between the connectors of the cleaning tubes and the ferrules of the endoscope due to dirt.
(1) Liquid feeding is performed simultaneously from the cleaning tubes A and B.
(2) Liquid feeding is performed only from the cleaning tube A (or a small amount of liquid is fed simultaneously from the cleaning tube B).
(3) Liquid feeding is performed only from the cleaning tube B (or a small amount of liquid is fed simultaneously from the cleaning tube A).

A clogging of the gap between the ferrules of the endoscope and the connectors of the cleaning tubes due to dirt is caused by a fluid containing the dirt flowing reversely to flow out from the gap.

### SUMMARY

According to aspects of the present disclosure, a reprocessor is provided, which includes a first tube, a second tube, a supplying apparatus, and a processor. The first tube is connectable to a first conduit of medical equipment with a gap therebetween. The second tube is connectable to a second conduit of the medical equipment. The supplying apparatus is configured to supply a fluid including a liquid to the first conduit via the first tube and to supply the fluid to the second conduit via the second tube. The processor is configured to control the supplying apparatus to start supplying the fluid to the second conduit at a first timing. The processor is further configured to start supplying the fluid to the first conduit at a second timing before the fluid supplied to the second conduit flows out from the gap via a merging point and the first conduit, thereby pushing the fluid back toward the merging point from the first conduit. The medical equipment includes an internal conduit including the first conduit, the second conduit, and a third conduit, each of the first to third conduits being open at one end and merging with the other two conduits at the merging point at another end.

According to aspects of the present disclosure, further provided is a method implementable by a reprocessor. The reprocessor includes a first tube connectable to a first conduit of an endoscope with a gap therebetween, and a second tube connectable to a second conduit of the endoscope. The method includes starting to supply a fluid to the second conduit via the second tube at a first timing. The method further includes starting to supply the fluid to the first conduit via the first tube at a second timing before the fluid supplied to the second conduit flows out from the gap via a merging point and the first conduit, thereby pushing the fluid back toward the merging point from the first conduit. The endoscope includes an internal conduit including the first conduit, the second conduit, a third conduit having an opening at a distal end of an insertion portion of the endoscope, the first, second, and third conduits merging with each other at the merging point.

According to aspects of the present disclosure, further provided is a non-transitory computer-readable medium storing computer-readable instructions executable by a processor of a reprocessor. The reprocessor includes a first tube connectable to a first conduit of an endoscope with a gap therebetween, and a second tube connectable to a second conduit of the endoscope. The computer-readable instructions are configured to, when executed by the processor, cause the reprocessor to start supplying a fluid to the second conduit via the second tube at a first timing. The computer-readable instructions are further configured to, when executed by the processor, cause the reprocessor to start supplying the fluid to the first conduit via the first tube at a second timing before the fluid supplied to the second conduit flows out from the gap via a merging point and the first conduit, thereby pushing the fluid back toward the merging point from the first conduit. The endoscope includes an internal conduit including the first conduit, the second conduit, a third conduit having an opening at a distal end of an insertion portion of the endoscope, the first, second, and third conduits merging with each other at the merging point.

### BRIEF DESCRIPTION OF DRAWINGS

FIG. 1 is a perspective view showing a configuration of a reprocessor for medical equipment according to a first embodiment of the present disclosure.

FIG. 2 shows a configuration related to fluid control of the reprocessor for medical equipment according to the first embodiment.

FIG. 3 shows a configuration example of a connecting part between a connector of a first tube of the reprocessor for medical equipment according to the first embodiment and a forceps plug ferrule.

FIG. 4 shows a flow of a fluid fed from a suction ferrule to an internal conduit of an endoscope in the first embodiment.

FIG. 5 is a chart for describing operation for preventing a gap of the connecting part between the connector of the first tube and the forceps plug ferrule from being clogged with dirt due to the fluid fed from the suction ferrule, in the first embodiment.

FIG. 6 is a timing chart showing operation for feeding a liquid from a second tube, and then feeding the liquid from the first tube and the second tube in the first embodiment.

FIG. 7 is a timing chart showing a first example of the fluid control in a case where a position where a fluid supplied from the first tube and a fluid supplied from the second tube collide with each other is located on a side closer to the forceps plug ferrule relative to a merging point, in the first embodiment.

FIG. 8 is a timing chart showing a second example of the fluid control in a case where the position where the fluid supplied from the first tube and the fluid supplied from the second tube collide with each other is located on a side closer to the suction ferrule relative to the merging point, in the first embodiment.

FIG. 9 is a flowchart showing an example of fluid control processing in the reprocessor for medical equipment in the first embodiment.

### DESCRIPTION OF EMBODIMENTS

Hereinafter, an embodiment of the present disclosure will be described with reference to drawings. However, the present disclosure is not limited by the embodiment to be described below.

Note that in the description in the drawings, the same or corresponding elements are attached with the same reference signs as appropriate. In addition, the drawings are schematic, and care should be taken to the fact that a relationship among the lengths of the respective elements, a ratio of the lengths among the respective elements, the number of the respective elements, and the like in one drawing are sometimes different from the actual ones for simplification of the description. Furthermore, the respective drawings sometimes include parts in which the relationships and ratios among the lengths of the elements are different.

### First Embodiment

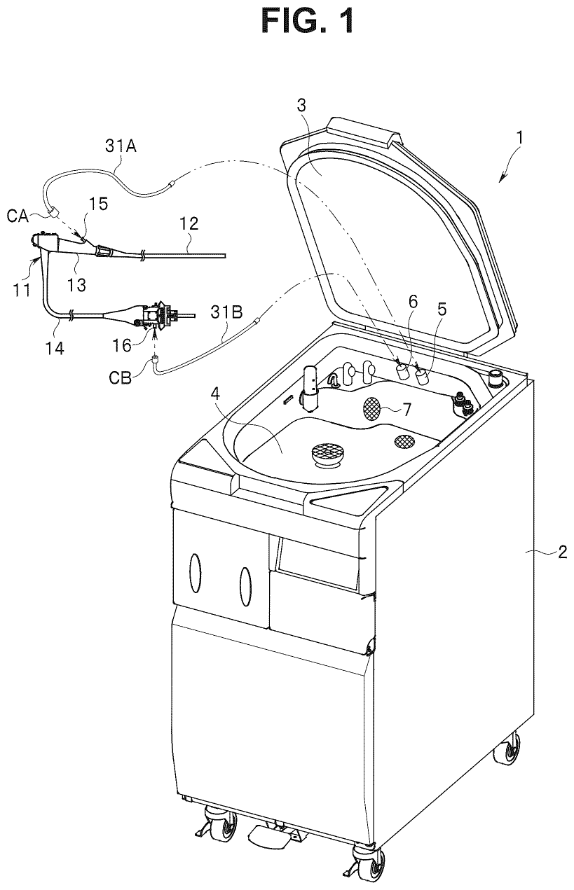

FIG. 1 to FIG. 9 show a first embodiment of the present disclosure. FIG. 1 is a perspective view showing a configuration of a reprocessor for medical equipment 1 according to the first embodiment.

Note that, in the present embodiment, description will be made by taking an endoscope 11 as medical equipment to be reprocessed, as an example. In this case, the reprocessor for medical equipment 1 is a reprocessor for an endoscope. However, the medical equipment to be reprocessed is not limited to the endoscope 11. The reprocessor for medical equipment of the present disclosure can be widely applied to medical equipment provided with three conduits (included in an internal conduit) each configured to open at one end side and merge at the other end side at a merging point.

As shown in FIG. 1, the reprocessor for medical equipment 1 includes an apparatus main body 2, and a top cover 3. The top cover 3 is openable/closable with respect to the apparatus main body 2. FIG. 1 shows a state where the top cover 3 of the reprocessor for medical equipment 1 is open.

The reprocessor for medical equipment 1 is an apparatus for reprocessing the endoscope 11 after use so as to bring the endoscope 11 into a reusable state. The reprocessing is not limited to specific processing, but may be any of rinsing with water, cleaning for removing dirt such as organic matters including protein, disinfection for disabling certain microorganisms, and sterilization for eliminating or killing all the microorganisms, or a combination of two or more of these.

The apparatus main body 2 includes, at an upper portion thereof, a processing tank 4 in which the endoscope 11 to be reprocessed is housed. The processing tank 4 is configured to be blocked from outside by closing the top cover 3.

The endoscope 11 includes an insertion portion 12, an operation portion 13, and a universal cord 14. The insertion portion 12 is a part configured to be inserted into a subject. The operation portion 13 is disposed on the proximal end side of the insertion portion 12, and is used by an operator to operate the endoscope 11. The universal cord 14 is extended, for example, from a lateral surface on the proximal end side of the operation portion 13, and connected, at the extension end thereof, to an image processor, a light source apparatus, and the like.

The operation portion 13 includes, on the distal end side thereof, a forceps plug ferrule 15 which is a proximal-end-side opening of a treatment instrument channel. The universal cord 14 includes, at the extension end thereof, a suction ferrule 16 which is a proximal-end-side opening of a suction channel.

The processing tank 4 stores, after the endoscope 11 is arranged therein, liquid such as a cleaning solution (for instance, alcohol or a disinfectant solution; hereinafter collectively referred to as the cleaning solution), water, etc. The cleaning solution is supplied from a tank in the apparatus main body 2. Water is supplied from a water faucet, not shown, via a water feeding hose connected to the reprocessor for medical equipment 1.

The processing tank 4 includes inside thereof a circulation port 7 communicated with a liquid pump 21 to be described later. The circulation port 7 is provided with a mesh filter, for example, and configured to filter dirt such as organic matters contained in the liquid such as the cleaning solution.

The processing tank 4 is further provided with a forceps plug port 5 and a suction ferrule port 6. The forceps plug port 5 is a port to which one end of a first tube 31A is connected. The suction ferrule port 6 is a port to which one end of a second tube 31B is connected.

The first tube 31A includes, on the other end thereof, a connector CA. The connector CA is connected to the forceps plug ferrule 15 of the endoscope 11. The forceps plug port 5 is communicated with the forceps plug ferrule 15 of the endoscope 11, via the first tube 31A. Thus, the first tube 31A is connected to the treatment instrument channel which is the internal conduit of the endoscope 11, via the connector CA and the forceps plug ferrule 15.

The second tube 31B includes, on the other end thereof, a connector CB. The connector CB is connected to the suction ferrule 16 of the endoscope 11. The suction ferrule port 6 is communicated with the suction ferrule 16 of the endoscope 11, via the second tube 31B. Thus, the second tube 31B is connected to the suction channel which is the internal conduit of the endoscope 11, via the connector CB and the suction ferrule 16.

When the endoscope 11 is reprocessed by the reprocessor for medical equipment 1, the top cover 3 is opened and the endoscope 11 is arranged in the processing tank 4. Then, the first tube 31A is connected to the forceps plug port 5 and the forceps plug ferrule 15. Furthermore, the second tube 31B is connected to the suction ferrule port 6 and the suction ferrule 16. Thus, the endoscope 11 and a fluid conduit of the reprocessor for medical equipment 1 is connected to each other. After setting the endoscope 11 in the processing tank 4, the top cover 3 is closed, thereby bringing the reprocessor into a reprocessing enabled state.

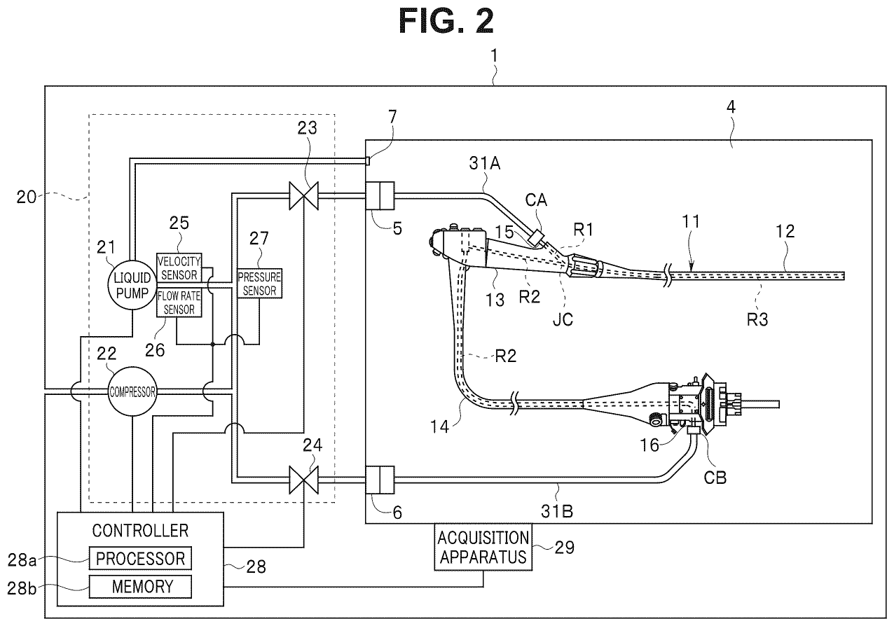

Next, FIG. 2 shows a configuration related to fluid control of the reprocessor for medical equipment 1 according to the first embodiment.

FIG. 2 shows a state where the endoscope 11 is connected to the reprocessor for medical equipment 1 by the first tube 31A and the second tube 31B. Note that, in FIG. 2, illustration of other configuration of the reprocessor for medical equipment 1 is omitted as appropriate.

In addition to the above-described processing tank 4, the forceps plug port 5, the suction ferrule port 6, and the circulation port 7, the reprocessor for medical equipment 1 includes: the liquid pump 21; a compressor 22; a first solenoid valve 23; a second solenoid valve 24; a velocity sensor 25; a flow rate sensor 26; a pressure sensor 27; a controller 28; and an acquisition apparatus 29.

The liquid pump 21, the compressor 22, the first solenoid valve 23, and the second solenoid valve 24 constitute a supplying apparatus 20 configured to supply a fluid to the internal conduit of the endoscope 11. Note that the velocity sensor 25, the flow rate sensor 26, and the pressure sensor 27 may further be included in the supplying apparatus 20. In addition, there is no need for providing all of the velocity sensor 25, the flow rate sensor 26, and the pressure sensor 27. One or more of these may be omitted. Alternatively, another sensor for detecting a physical quantity, other than the velocity sensor 25, the flow rate sensor 26, and the pressure sensor 27, may be further provided.

The supplying apparatus 20 can supply a fluid including a liquid to a first conduit R1, to be described later, of the endoscope 11, via the first tube 31A. Furthermore, the supplying apparatus 20 can supply the fluid to a second conduit R2, to be described later, of the endoscope 11, via the second tube 31B.

The liquid pump 21 takes in the liquid such as the cleaning solution in the processing tank 4 from the circulation port 7. The liquid pump 21 pressurizes the taken-in liquid to a predetermined pressure, and feeds the pressurized liquid. A liquid-feeding path of the liquid is branched into two parts, and one of the two parts is connected to the forceps plug port 5 via the first solenoid valve 23 and the other of the two parts is connected to the suction ferrule port 6 via the second solenoid valve 24.

The compressor 22 functions as a gas pump. The compressor 22 takes in a gas, for example, air, to pressurize the gas, and feeds the pressurized gas. A gas-feeding path of the gas is branched into two parts, and one of the two parts is connected to the forceps plug port 5 via the first solenoid valve 23 and the other of the two parts is connected to the suction ferrule port 6 via the second solenoid valve 24.

In other words, the liquid-feeding path from the liquid pump 21 and the gas-feeding path from the compressor 22 share a path after their respective branch points.

Employing such a configuration makes it possible for the supplying apparatus 20 to supply only the liquid to the internal conduit of the endoscope 11 by driving the liquid pump 21 and stopping the compressor 22. Furthermore, the supplying apparatus 20 can supply only the gas to the internal conduit of the endoscope 11 by stopping the liquid pump 21 and driving the compressor 22. Furthermore, the supplying apparatus 20 can supply a fluid in which the liquid and the gas are mixed to the internal conduit of the endoscope 11 by driving both the liquid pump 21 and the compressor 22.

The first solenoid valve 23 can be switched to an open state or a closed state according to a control signal transmitted from the controller 28. When the first solenoid valve 23 is brought into the open state, the liquid pump 21 and the compressor 22 are communicated with the forceps plug ferrule 15 of the endoscope 11.

The second solenoid valve 24 can be switched to an open state or a closed state according to the control signal transmitted from the controller 28. When the second solenoid valve 24 is brought into the open state, the liquid pump 21 and the compressor 22 are communicated with the suction ferrule 16 of the endoscope 11.

The velocity sensor 25, the flow rate sensor 26, and the pressure sensor 27 detect physical quantities related to the fluid that is supplied to the second conduit R2 by the supplying apparatus 20.

The velocity sensor 25 is arranged, for example, in the vicinity of the liquid pump 21 so as to be located at a position between the liquid pump 21 and the branch point of the liquid-feeding path. The velocity sensor 25 is configured to detect a velocity of the liquid fed from the liquid pump 21. The velocity sensor 25 transmits a detection result of the velocity of the liquid to the controller 28. For example, when the first solenoid valve 23 is in the closed state and the second solenoid valve 24 is in the open state, the velocity sensor 25 detects the velocity of the liquid (fluid) that is supplied to the second conduit R2.

The flow rate sensor 26 is arranged, for example, in the vicinity of the liquid pump 21 so as to be located at a position between the liquid pump 21 and the branch point of the liquid-feeding path. The flow rate sensor 26 is configured to detect an amount of the liquid (flow rate) that is fed per unit time from the liquid pump 21. The flow rate sensor 26 transmits a detection result of the flow rate of the liquid to the controller 28. For example, when the first solenoid valve 23 is in the closed state and the second solenoid valve 24 is in the open state, the flow rate sensor 26 detects the flow rate of the liquid (fluid) that is supplied to the second conduit R2.

The pressure sensor 27 is arranged, for example, at the branch point of the liquid-feeding path from the liquid pump 21. The pressure sensor 27 is configured to detect a pressure at the branch point of the liquid fed from the liquid pump 21. The pressure sensor 27 transmits a detection result of the pressure at the branch point to the controller 28. For example, when the first solenoid valve 23 is in the closed state and the second solenoid valve 24 is in the open state, the pressure sensor 27 can substantially detect the pressure of the liquid (fluid) that is supplied to the second conduit R2.

The controller 28 includes a processor 28a and a memory 28b. The processor 28a may include at least one of an ASIC (Application Specific Integrated Circuit), an FPGA (Field Programmable Gate Array), or a CPU (Central Processing Unit). The memory 28b is a storage medium including, for example, a non-volatile memory such as a ROM and a volatile memory such as a RAM. The processor 28a reads and executes a processing program stored in the non-volatile memory of the memory 28b to achieve various functions of the controller 28. However, the configuration of the controller 28 is not limited to the above. For example, one or more constituent parts that achieve various functions of the controller 28 may be configured as a dedicated electronic circuit.

The controller 28 is electrically connected to the liquid pump 21, the compressor 22, the first solenoid valve 23, the second solenoid valve 24, the velocity sensor 25, the flow rate sensor 26, the pressure sensor 27, and the acquisition apparatus 29.

The controller 28 acquires information related to the internal conduit of the endoscope 11 (conduit information) based on a signal acquired by the acquisition apparatus 29. The acquisition apparatus 29 may be any device, such as an RFID reader, an image sensor, a touch panel, or the like, as long as it is a device that can acquire signals.

The memory 28b stores, in advance, a table in which a model number, a production number, and the like of the endoscope 11 are associated with the conduit information such as a length, a diameter, etc., of the internal conduit of the endoscope 11. Note that the table can be updated to the latest one in a maintenance operation and the like.

When an image sensor is used as the acquisition apparatus 29, for example, the image sensor acquires an image of the endoscope 11 arranged in the processing tank 4. The processor 28a reads an image signal acquired by the image sensor and performs an image analysis, to estimate the model number, the production number, etc., of the endoscope 11. Note that the processor 28a may estimate the length, etc., of the internal conduit of the endoscope 11 by the image analysis.

When an RFID reader is used as the acquisition apparatus 29, for example, the RFID reader reads information of an RF tag attached to the endoscope 11. The RF tag stores the model number, the production number, etc., of the endoscope 11. When a touch panel is used as the acquisition apparatus 29, for example, the user operates the touch panel to input the model number, the production number, etc., of the endoscope 11. Alternatively, the user may operate the touch panel to input the conduit information of the endoscope 11.

The processor 28a refers to the table in the memory 28b, based on the information such as the model number, the production number, etc., of the endoscope 11 included in the signal read from the acquisition apparatus 29, or the information such as the model number, the production number, etc., which have been estimated by the image analysis, and acquires the conduit information such as the length and the diameter of the internal conduit of the endoscope 11. Alternatively, the processor 28a acquires the conduit information estimated by the image analysis or the conduit information inputted through the touch panel.

The processor 28a controls, based on the conduit information of the endoscope 11 and one or more of the velocity, the flow rate, and the pressure of the liquid, which are acquired respectively from the velocity sensor 25, the flow rate sensor 26, and the pressure sensor 27, the driving of the liquid pump 21, the driving of the compressor 22, the opening/closing operation of the first solenoid valve 23, and the opening/closing operation of the second solenoid valve 24. Thus, the processor 28a controls the supplying apparatus 20.

The endoscope 11 includes the treatment instrument channel and the suction channel, as described above. The treatment instrument channel and the suction channel are connected to each other at a merging point JC. A part of the treatment instrument channel, which is from the forceps plug ferrule 15, which is an opening, to the merging point JC, is referred to as the first conduit R1. A part of the treatment instrument channel, which is from the merging point JC to the distal-end-opening of the insertion portion 12, is referred to as a third conduit R3. A part of the suction channel, which is from the suction ferrule 16, which is an opening, to the merging point JC, is referred to as the second conduit R2. The third conduit R3 serves both as the treatment instrument channel and the suction channel.

Thus, the endoscope 11 includes the internal conduit including the first conduit R1, the second conduit R2, and the third conduit R3. The first conduit R1, the second conduit R2, and the third conduit R3 are each open at one end side and merge at the other end side at the merging point JC.

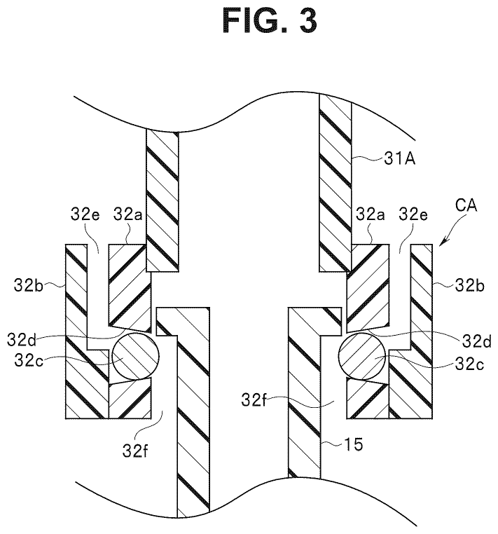

FIG. 3 shows a configuration example of a connecting part between the connector CA of the first tube 31A of the reprocessor for medical equipment 1 according to the first embodiment and the forceps plug ferrule 15.

The connector CA of the first tube 31A includes a connector main body 32a, a connector cover 32b, and a plurality of spherical bodies 32c. The connector main body 32a is provided at the distal end of the first tube 31A. The connector cover 32b is provided on the outer circumferential portion of the connector main body 32a. The plurality of spherical bodies 32c are arranged substantially between the connector main body 32a and the connector cover 32b.

The connector main body 32a has a cylindrical shape, and includes a plurality of circular holes 32d provided at equiangular positions on the circumferential lateral portion of the connector main body 32a. The circular holes 32d are arranged such that, for example, four circular holes are located at 90 degree intervals along the circumferential direction on the circumferential lateral portion of the connector main body 32a. Each of the circular holes 32d has a tapered cross section such that a diameter thereof is reduced from the outer circumferential surface toward the inner circumferential surface of the connector main body 32a.

The diameter of each of the plurality of spherical bodies 32c is larger than the diameter of each of the circular holes 32d on the inner circumferential surface of the circumferential lateral portion. With such a configuration, each of the spherical bodies 32c can partly protrude from the inner circumferential side opening of each of the circular holes 32d, but does not fall from the inner circumferential surface of the connector main body 32a.

The connector cover 32b is arranged further outward on the outer circumferential side of the connector main body 32a than the plurality of spherical bodies 32c. A circumferential gap 32e is formed between the connector cover 32b and the connector main body 32a.

The forceps plug ferrule 15 is formed in a cylindrical shape, and includes, at the distal end thereof, an outward flange. The connector CA is detachably attached to the forceps plug ferrule 15.

In the state where the connector CA is attached to the forceps plug ferrule 15, each of the plurality of spherical bodies 32c partly protrudes from the inner circumferential side opening of each of the circular holes 32d, to lock the outward flange of the forceps plug ferrule 15 so as to prevent falling off.

A circumferential gap 32f is formed between the outer circumferential surface of the forceps plug ferrule 15 and the inner circumferential surface of the connector main body 32a.

The fluid such as the cleaning solution fed from the first tube 31A flows out from the gap 32f, via the part between the connector main body 32a and the forceps plug ferrule 15, and further flows out from the gap 32e, via the space between the circular holes 32d and the spherical bodies 32c, to enable sufficient cleaning of the forceps plug ferrule 15.

Note that the shape of the connector CA shown in FIG. 3 is one example, and the connector CA having another shape may be used as long as the fluid can be flowed out from the gap in the connecting part between the connector CA and the forceps plug ferrule 15.

Thus, the first tube 31A and the first conduit R1 are connected to each other, with the gaps 32e and 32f that are formed between the connector CA and the forceps plug ferrule 15. The gaps 32e and 32f are provided so that a connecting surface with the connector CA of the forceps plug ferrule 15 can also be cleaned sufficiently.

Note that the configuration in FIG. 3 has been described above, as the configuration example of the connecting part between the connector CA and the forceps plug ferrule 15. However, the connecting part between the connector CB and the suction ferrule 16 is also similarly configured with gaps, to thereby enable a fluid to be flowed out from the gaps. Such a configuration is intended for sufficient cleaning of the connecting surface with the connector CB of the suction ferrule 16.

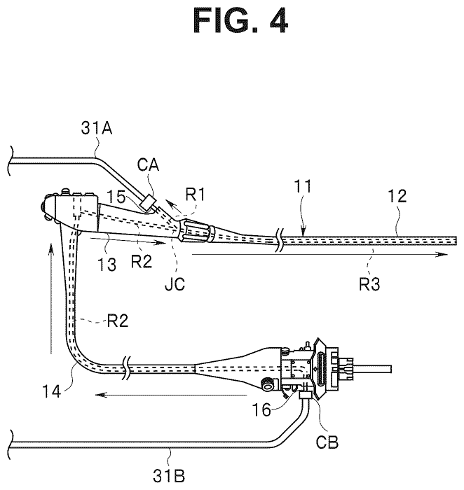

FIG. 4 shows a flow of the fluid fed from the suction ferrule 16 to the internal conduit of the endoscope 11 in the first embodiment.

The processor 28a brings the first solenoid valve 23 into the closed state and the second solenoid valve 24 into the open state, to drive at least the liquid pump 21 (may drive also the compressor 22 as needed). With such driving, the fluid including the liquid is supplied from the second tube 31B to the internal conduit of the endoscope 11.

Then, the fluid including the liquid flows through the second conduit R2, reaches the merging point JC, where it branches. In other words, the fluid that has reached the merging point JC flows such that a part thereof flows into the third conduit R3 and another part thereof flows into the first conduit R1. If the fluid flowed into the first conduit R1 reaches the connector CA, the fluid flows out from the gaps 32e and 32f of the connector CA.

Incidentally, the suction channel is used for performing suction from the inside of the subject. Therefore, dirt such as organic matters sometimes adheres to the inside of the second conduit R2 of the endoscope 11 after use. The fluid including the liquid, that is, only the liquid or a gas-liquid mixed fluid in which the liquid and the gas are mixed can convey the dirt such as the organic matters (Note that it may be practically considered that the fluid including only the gas does not convey the dirt such as the organic matters.)

If the fluid containing dirt flows out from the gaps 32e and 32f of the connector CA, the gaps 32e and 32f may become clogged. In that case, the flow of the fluid from the gaps 32e and 32f is blocked, which results in insufficient cleaning of the forceps plug ferrule 15.

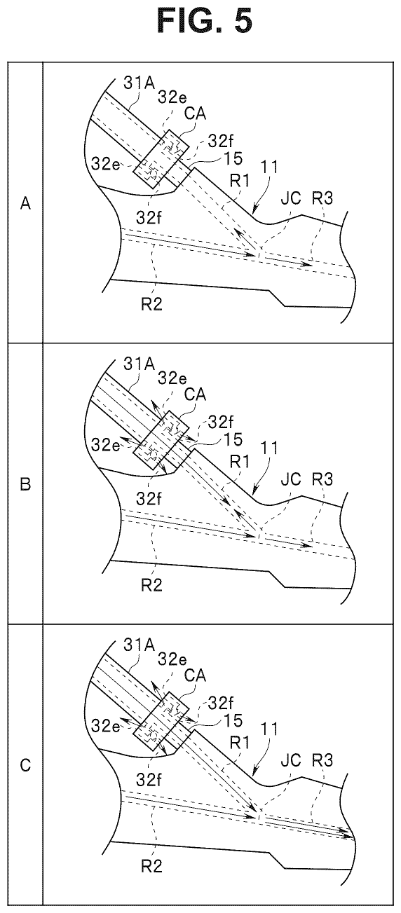

In view of the above, the controller 28 in the present embodiment performs the fluid control as shown in FIG. 5. FIG. 5 is a chart for describing operation for preventing the gaps 32e and 32f of the connecting part between the connector CA of the first tube 31A and the forceps plug ferrule 15 from being clogged with the dirt due to the fluid fed from the suction ferrule 16, in the first embodiment.

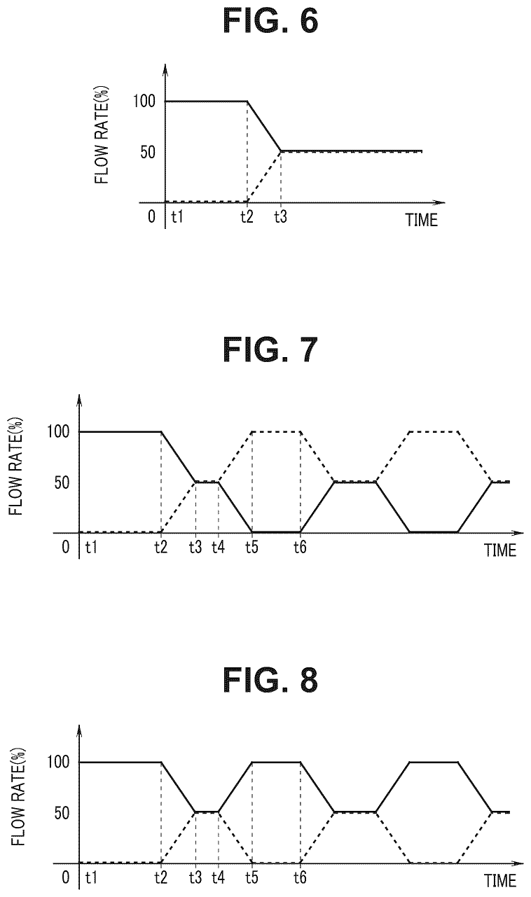

FIG. 6 is a timing chart showing the operation for feeding the liquid from the second tube 31B and then feeding the liquid from the first tube 31A and the second tube 31B in the first embodiment. In FIG. 6 (and similarly in FIGS. 7 and 8 to be described later), the solid line indicates the flow rate of the fluid flowing through the second conduit R2, and the dotted line indicates the flow rate of the fluid flowing through the first conduit R1.

The liquid pump 21 is driven with a constant power, to feed the fluid at a predetermined flow rate. The supplying apparatus 20 supplies the fluid at the predetermined flow rate, which is fed from the liquid pump 21, to either the first conduit R1 or the second conduit R2, or distributes to supply the fluid to the first conduit R1 and the second conduit R2. The vertical axis in FIG. 6 shows the flow rate expressed with a percentage (%) relative to the predetermined flow rate.

The used endoscope 11 is set in the processing tank 4, and the top cover 3 is closed, to start the reprocessing. Then, under the control by the controller 28, the processing tank 4 is filled with the cleaning solution.

Even after the processing tank 4 is filled with the cleaning solution, air is remained in the internal conduit of the endoscope 11, and the internal conduit is not filled with the liquid.

In this state, the controller 28 brings the first solenoid valve 23 into the closed state and the second solenoid valve 24 into the open state, to drive the liquid pump 21. Then, the fluid is fed via the suction ferrule port 6 and the second tube 31B, to flow from the second tube 31B into the second conduit R2 at a first timing (the time t1 in FIG. 6, hereinafter, referred to as the first timing t1 as appropriate).

Thus, the supply of the fluid to the second conduit R2 is started at the first timing t1 without supplying the fluid to the first conduit R1.

Note that the controller 28 can estimate which time point after the start of the driving of the liquid pump 21 becomes the first timing at which the fluid flows into the second conduit R2, since the length and diameter of the conduit in the reprocessor for medical equipment 1 and the length and diameter of the conduit of the second tube 31B are known.

The column A in FIG. 5 shows the state where the fluid fed from the second tube 31B and the suction ferrule 16 flows through the second conduit R2 to reach the merging point JC, and a part of the fluid is gradually flowing from the merging point JC to the first conduit R1.

The processor 28a estimates how much time elapses from the first timing t1 at which the supply of the fluid to the second conduit R2 has been started, before a third timing (for example, the time t3 shown in FIG. 6, hereinafter referred to as the third timing t3 as appropriate) occurs. At the third timing, the supplied fluid is started flowing from the gaps 32e and 32f of the connector CA, via the merging point JC and the first conduit R1.

The processor 28a calculates, based on the physical quantity detected by one or more sensors among the velocity sensor 25, the flow rate sensor 26, and the pressure sensor 27, the position of the fluid, the supply of which to the second conduit R2 has been started at the first timing t1, in the internal conduit. Then, the processor 28a estimates the third timing t3 based on the calculated position of the fluid.

A first method for estimating the third timing t3 is a method based on the detection value of the velocity sensor 25. In other words, the processor 28a estimates the third timing t3 based on the velocity of the fluid detected by the velocity sensor 25, the length of the first conduit R1, and the length of the second conduit R2.

A second method for estimating the third timing t3 is a method based on the detection value of the flow rate sensor 26. In other words, the processor 28a estimates the third timing t3 based on the flow rate of the fluid detected by the flow rate sensor 26, the length and the cross-sectional area of the first conduit R1, and the length and the cross-sectional area of the second conduit R2. Note that the cross-sectional area is calculated based on the diameter of each of the conduits. In addition, the volume of the inside of the conduit is calculated by multiplying the length by the cross-sectional area.

A third method for estimating the third timing t3 is a method based on the detection value of the pressure sensor 27. In this case, the memory 28b stores, in advance, data indicating a relationship between the pressure of the fluid detected by the pressure sensor 27 and the head position (hereinafter may be referred to as the leading-end position) of the fluid flowing through the first conduit R1 and the second conduit R2. Based on the pressure of the fluid detected by the pressure sensor 27, the processor 28a refers to the memory 28b to estimate the third timing t3.

The processor 28a estimates a second timing (for example, the time t2 shown in FIG. 6, hereinafter, referred to as the second timing t2 as appropriate). The estimation of the second timing t2 is performed by setting the second timing t2 after the first timing t1 and before the estimated third timing t3. Accordingly, the second timing t2 can be set to an appropriate timing as long as it is after the first timing t1 and before the third timing t3.

Then, the processor 28a controls the supplying apparatus 20 so as to start the supply of the fluid to the first conduit R1 at the second timing t2.

The column B in FIG. 5 shows the state where, before the third timing t3 at which the fluid fed from the suction ferrule 16 reaches the gaps 32e and 32f of the connector CA, via the merging point JC, the processor 28a has brought the first solenoid valve 23 into the open state to cause the fluid to be fed from the first tube 31A and the forceps plug ferrule 15 and the supply of the fluid to the first conduit R1 has started at the second timing t2.

When the fluid fed from the first tube 31A reaches the connector CA, the fluid flows out from the gaps 32e and 32f. Since the fluid fed from the first tube 31A does not contain dirt, there is no possibility that the gaps 32e and 32f are clogged with dirt. The forceps plug ferrule 15 is cleaned sufficiently by the fluid that flows out from the gaps 32e and 32f.

The column C in FIG. 5 shows the state where the fluid fed from the suction ferrule 16 is pushed back toward the merging point JC side by the fluid fed from the forceps plug ferrule 15, and moves toward the third conduit R3 side via the merging point JC. The fluid fed from the suction ferrule 16 is pushed back before reaching the connector CA, which eliminates the possibility that the gaps 32e and 32f are clogged with dirt.

Incidentally, after the time t3 shown in FIG. 6, the fluid at the predetermined flow rate, which is supplied by the supplying apparatus 20, is distributed by 50%, for example, to each of the first conduit R1 and the second conduit R2 (another rate may be applied as long as the total amount is 100%).

A position where the fluid supplied from the first tube 31A and the fluid supplied from the second tube 31B collide with each other in the internal conduit is hereinafter referred to as a collision position. For example, in the column B in FIG. 5, the collision position is shown as the position where the arrows mutually oppositely directed collide with each other on the first conduit R1.

The collision position is located on the first conduit R1 in some cases, and on the second conduit R2 in other cases. Description will be made on a fluid control method in the case where the collision position is located on the first conduit R1, with reference to FIG. 7. In addition, description will be made later on a fluid control method in the case where the collision position is located on the second conduit R2, with reference to FIG. 8.

In the control in which the fluid supply from the second tube 31B is started at the time t1, and thereafter the fluid supply from the first tube 31A is started at the time t2, when the collision position is located on the first conduit R1, the second conduit R2 is cleaned at the flow rate of 100%, but the first conduit R1 is cleaned only at the flow rate of 50%. In view of the above, the fluid control method shown in FIG. 7 is applied, to enable also the first conduit R1 to be cleaned at the flow rate of 100%.

FIG. 7 is a timing chart showing a first example of the fluid control in the case where the position where the fluid supplied from the first tube 31A and the fluid supplied from the second tube 31B collide with each other is located on a side closer to the forceps plug ferrule 15 relative to the merging point JC, in the first embodiment.

The processor 28a performs the fluid control, from the time t3 to t4, for example, at the flow rate of 50% for the first conduit R1 and at the flow rate of 50% for the second conduit R2. After that, the processor 28a drives the liquid pump 21 and brings the second solenoid valve 24 into the closed state, with the first solenoid valve 23 remaining in the open state.

Then, the flow rate of the fluid that is supplied from the supplying apparatus 20 to the first conduit R1 increases, and the flow rate of the fluid that is supplied from the supplying apparatus 20 to the second conduit R2 decreases. Then, at the time t5 to t6, the flow rate becomes 100% for the first conduit R1 and 0% for the second conduit R2.

However, if the state is continued as it is, the fluid supplied from the supplying apparatus 20 to the first conduit R1 flows backward through the second conduit R2 to reach the connector CB, which sometimes results in the clogging of the gap of the connector CB with the dirt contained in the fluid flowing out from the gap of the connector CB.

To address the circumstance, the second solenoid valve 24 is brought into the open state again at the time t6 which is before the fluid supplied from the supplying apparatus 20 to the first conduit R1 reaches the gap of the connector CB, to thereby prevent the gap of the connector CB from being clogged with the dirt.

After that, similarly, fluid control is performed by repeating a first state where the flow rate for the first conduit R1 is 50% and the flow rate for the second conduit R2 is 50% and a second state where the flow rate for the first conduit R1 is 100% and the flow rate for the second conduit R2 is 0%. Note that the ratio of the flow rate for the first conduit R1 and the flow rate for the second conduit R2 may be changed each time the fluid control is performed by repeating the first state and the second state (that is, the ratio does not have to be the same).

Such repeating is performed, at the second timing t2 or after, by the processor 28a controlling the first solenoid valve 23 and the second solenoid valve 24 such that the fluid at the predetermined flow rate, which is supplied from the supplying apparatus 20, is supplied continuously to the first conduit R1 and intermittently to the second conduit R2.

Next, in the control in which the fluid supply from the second tube 31B is started at the time t1, and thereafter the fluid supply from the first tube 31A is started at the time t2, when the collision position is located on the second conduit R2, a part of the second conduit R2, which is from the suction ferrule 16 to the collision position, is cleaned at the flow rate of 100%, but another part of the second conduit R2, which is from the collision position to the merging point JC, and the first conduit R1 are cleaned at the flow rate of only 50%. In view of the above, the fluid control method shown in FIG. 8 is applied, to thereby enable a longer part of the conduit to be cleaned at the flow rate of 100%.

FIG. 8 is a timing chart showing a second example of the fluid control in the case where the position where the fluid supplied from the first tube 31A and the fluid supplied from the second tube 31B collide with each other is located on a side closer to the suction ferrule 16 relative to the merging point JC, in the first embodiment.

The processor 28a performs the fluid control, at the time t3 to t4, for example, at the flow rate of 50% for the first conduit R1 and at the flow rate of 50% for the second conduit R2. After that, the processor 28a drives the liquid pump 21, and brings the first solenoid valve 23 into the closed state, with the second solenoid valve 24 remaining in the open state.

Then, the flow rate of the fluid that is supplied from the supplying apparatus 20 to the first conduit R1 decreases, and the flow rate of the fluid that is supplied from the supplying apparatus 20 to the second conduit R2 increases. Then, at the time t5 to t6, the flow rate becomes 0% for the first conduit R1 and 100% for the second conduit R2.

However, if the state is continued as it is, the fluid supplied from the supplying apparatus 20 to the second conduit R2 flows backward through the first conduit R1 to reach the connector CA, which sometimes results in the clogging of the gaps 32e and 32f of the connector CA with the dirt contained in the fluid flowing out from the gaps 32e and 32f of the connector CA.

To address the circumstance, the first solenoid valve 23 is brought into the open state again at the time t6 which is before the fluid supplied from the supplying apparatus 20 to the second conduit R2 reaches the gaps 32e and 32f of the connector CA, to thereby prevent the gaps 32e and 32f of the connector CA from being clogged with the dirt.

After that, similarly, fluid control is performed by repeating the first state where the flow rate for the first conduit R1 is 50% and the flow rate for the second conduit R2 is 50%, and a third state where the flow rate for the first conduit R1 is 0% and the flow rate for the second conduit R2 is 100%.

Such repeating is performed, at the second timing t2 or after, by the processor 28a controlling the first solenoid valve 23 and the second solenoid valve 24 such that the fluid at the predetermined flow rate, which is supplied from the supplying apparatus 20, is supplied continuously to the second conduit R2 and intermittently to the first conduit R1.

However, in this case, the second conduit R2 can be cleaned at the flow rate of 100%, while the first conduit R1 can only be cleaned at the flow rate of 50%. In view of the above, it may be preferable to not only repeat the first state and the third state but also further include the second state where the flow rate for the first conduit R1 is 100% and the flow rate for the second conduit R2 is 0% as shown in FIG. 7, thereby enabling the first conduit R1 to be also cleaned at the flow rate of 100%.

In general, under the same flow rate, the longer the time period during which the fluid is flowed, the greater the cleaning effect. Therefore, if the state where the cleaning is performed at the flow rate of 100% cannot be achieved, the sufficient cleaning effect may be obtained by extending the cleaning time.

Note that description has been made above mainly on the example of the control in which the fluid supply to the suction channel is started first, and after that the fluid supply to the treatment instrument channel is started.

However, a control can be performed in which the fluid supply to the treatment instrument channel is started first, and after that the fluid supply to the suction channel is started. In this case, such a control can be applied substantially, if the above description in which the part between the merging point JC and the forceps plug ferrule 15 is referred to as the first conduit R1 and the part between the merging point JC and the suction ferrule 16 is referred to as the second conduit R2 is interpreted as a description in which the part between the merging point JC and the suction ferrule 16 is referred to as the first conduit R1 and the part between the merging point JC and the forceps plug ferrule 15 is referred to as the second conduit R2.

When reprocessing starts, the endoscope 11 is cleaned by the cleaning solution in a first process. After the first process is performed, the cleaning solution is discharged from the processing tank 4, and the liquid is removed from the internal conduit by feeding a gas from the compressor 22 to the internal conduit of the endoscope 11. After that, in a second process, the endoscope 11 is rinsed with water.

In such a step, both at the time of starting the first process and the time of starting the second process, the internal conduit of the endoscope 11 is not filled with a liquid. Therefore, the above-described process for preventing the gaps 32e and 32f from being clogged with the dirt can be applied to both the time of starting the first process and the time of starting the second process.

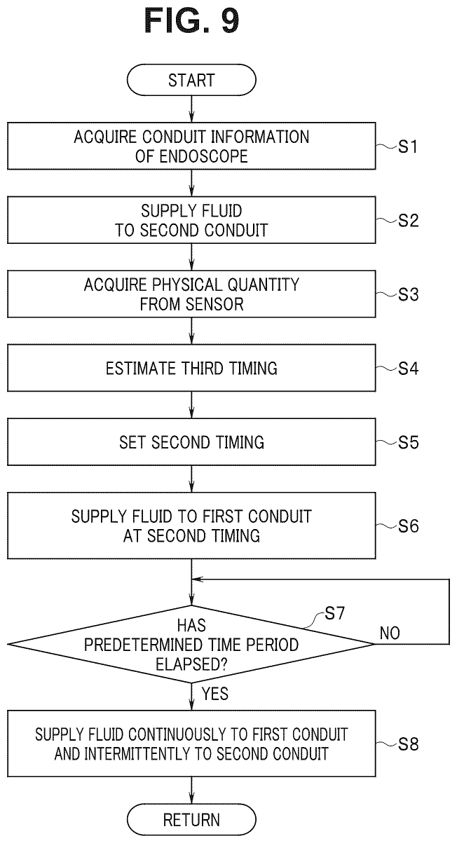

FIG. 9 is a flowchart showing an example of the fluid control processing in the reprocessor for medical equipment 1 in the first embodiment.

The processing shown in FIG. 9 is called and executed, for example, at each of the time of starting the first process and the time of starting the second process in the main process, not shown, of performing the processing of the entire reprocessor for medical equipment 1.

When the processing starts, the processor 28a acquires a signal from the acquisition apparatus 29, and acquires the conduit information of the endoscope 11 (Step S1). Then, the processor 28a brings the first solenoid valve 23 into the closed state, and the second solenoid valve 24 into the open state, and performs the supply of the fluid from the supplying apparatus 20 only to the second conduit R2 at the first timing t1 (Step S2).

After that, the processor 28a acquires a physical quantity from one or more sensors among the velocity sensor 25, the flow rate sensor 26, and the pressure sensor 27 (Step S3).

The processor 28a estimates, based on the acquired physical quantity, the third timing t3 at which the fluid supplied only to the second conduit R2 flows out from the gaps 32e and 32f, via the merging point JC and the first conduit R1 (Step S4).

The processor 28a sets the second timing t2 which is after the first timing t1 and before the third timing t3 (Step S5).

After that, at the time when the second timing t2 has come, the processor 28a switches the first solenoid valve 23 from the closed state to the open state, with the second solenoid valve 24 remaining in the open state, the fluid at the predetermined flow rate, which is supplied from the supplying apparatus 20, is distributed and supplied to the first conduit R1 and the second conduit R2 (Step S6).

After that, the processor 28a determines whether a predetermined time period (for example, the time period from the time t2 to the time t4 shown in FIG. 7) has elapsed (Step S7).

The processor 28a waits until the predetermined time period elapses, and when determining that the predetermined time period has elapsed in Step S7, the processor 28a controls the supplying apparatus 20 so as to supply the fluid continuously to the first conduit R1 and supply the fluid intermittently to the second conduit R2 as shown at the time t4 and after in FIG. 7, for example (Step S8).

After performing the processing in Step S8, the processor 28a returns the processing to the main process, not shown.

According to the first embodiment, before the fluid, which has been started to be supplied from the supplying apparatus 20 only to the second conduit R2, flows out from the gaps 32e and 32f between the connector CA and the forceps plug ferrule 15, the fluid is started to be supplied from the supplying apparatus 20 also to the first conduit R1. Such a configuration prevents the fluid containing the dirt from being flowed out from the gaps 32e and 32f, thereby preventing the gaps 32e and 32f from being clogged with the dirt. Therefore, the forceps plug ferrule 15 can be sufficiently reprocessed. Similarly, the suction ferrule 16 can also be sufficiently reprocessed.

Note that description has been made above mainly on the case where the present disclosure is the reprocessor for medical equipment. However, the present disclosure is not limited to the case. For example, the present disclosure may be a reprocessing method for medical equipment. In addition, the present disclosure may be a computer program for causing a computer to execute the same processing as that performed by the reprocessor for medical equipment. Furthermore, the present disclosure may be a non-transitory computer-readable (storage) medium that stores the computer program, or other media.

Some examples of the storage medium that stores a computer program product include a portable storage medium such as a flexible disk, a CD-ROM (compact disc read only memory), a DVD (digital versatile disc) or the like, or a storage medium such as an HDD (hard disk drive), an SSD (solid state drive), or the like. It is not limited to the entirety of the computer program to be stored in the storage medium, but a part of the computer program may be stored in the storage medium. The entirety or a part of the computer program may also be distributed or provided via a communication network. When a user installs the computer program on the computer from the storage medium or downloads the computer program via the communication network and installs it on the computer, the computer program is read by the computer and all or part of the operations are executed, to thereby enable the operations of the reprocessor for medical equipment to be executed.

In addition, the present disclosure is not limited as-is to the above described embodiment. Aspects of the present disclosure may be embodied by modifying the constituent elements within a range without departing from the gist of the disclosure at the practical stage. In addition, various aspects of the disclosure can be achieved by appropriately combining the plurality of constituent elements disclosed in the above-described embodiment. Some of the constituent elements may be deleted from all the constituent elements shown in the embodiment, for example. Furthermore, constituent elements over different embodiments may be combined as appropriate. It goes without saying that various modifications and applications can be implemented within a range without departing from the gist of the disclosure.

### General Interpretation Notes

The following applies throughout this specification and drawings.

It is noted that various connections are described between elements in the foregoing description. These connections, unless specified otherwise, may be either direct or indirect, and this specification is not intended to be limiting in that respect. Aspects of the present disclosure may be implemented using circuits (such as application-specific integrated circuits) or computer software stored on non-transitory computer-readable storage media, including but not limited to RAMs, ROMs, flash memories, EEPROMs, CD media, DVD media, temporary storage, hard disk drives, floppy drives, permanent storage, and the like.

As used herein, the term “processor” encompasses a single processor or a group of multiple processors, which may include a single-core processor, a multi-core processor, multiple processors within a single device, or multiple processors in wired or wireless communication with each other. Such processors may be locally or remotely distributed and may operate collaboratively or in a distributed fashion across a network of devices, the Internet, or the cloud to collectively perform the tasks attributed to the “processor” described herein. It should be understood that not all of the processors included in the system or device are necessarily involved in performing each operation attributed to the “processor.” Rather, only a subset of at least one processor may contribute to performing a particular operation. Furthermore, different subsets of at least one processor may contribute to performing different operations, and the composition of the subsets may vary from one operation to another. Similarly, the term “non-transitory computer-readable (storage) medium” encompasses a single storage medium or a group of multiple storage media, which may be locally or remotely distributed and may collectively store and provide access to instructions, data, or other information in a coordinated or distributed manner.

In the present disclosure, an inclusive OR—meaning that it includes either A, B, or both—may be expressed as “A and/or B,” “at least one of A or B,” or “at least one selected from the group consisting of A and B.” Additionally, the expressions “one of A or B” and “either A or B,” as used herein, refer to a case where A or B is selected exclusively, but not both. The same interpretation applies in cases where three or more selectable elements are considered.

While aspects of the present disclosure have been described in conjunction with specific illustrative embodiments and drawings, various modifications, alternatives, variations, improvements, and equivalent arrangements may be made by those skilled in the art without departing from the spirit or scope of the technical concepts according to aspects of the present disclosure. Accordingly, the embodiments described herein are intended to be illustrative, and not restrictive, of such technical concepts.

Non-limiting examples according to aspects of the present disclosure will be described in the following clauses:

Clause 1: A reprocessor for medical equipment, comprising:
a first tube configured to connect to a first conduit of the medical equipment with a gap, the medical equipment comprising an internal conduit that includes the first conduit, a second conduit, and a third conduit, the first conduit, the second conduit, and the third conduit each being open at one end side and merging at another end side at a merging point;
a second tube configured to connect to the second conduit;
a supplying apparatus configured to supply a fluid including a liquid to the first conduit via the first tube, and supply the fluid to the second conduit via the second tube; and
a processor configured to control the supplying apparatus, wherein
the processor is configured to control the supplying apparatus:
   to start supplying the fluid to the second conduit at a first timing; and
   to start supplying the fluid to the first conduit at a second timing before the fluid supplied to the second conduit flows out from the gap, via the merging point and the first conduit, and to push the fluid back from the first conduit toward the merging point.

Clause 2: The reprocessor for medical equipment according to clause 1, further comprising
a sensor configured to detect a physical quantity related to the fluid that is supplied to the second conduit by the supplying apparatus, wherein
the processor is configured to:
calculate, based on the physical quantity detected by the sensor, a position of the fluid, which has been started to be supplied to the second conduit at the first timing, in the internal conduit; and
estimate the second timing based on the calculated position of the fluid.

Clause 3: The reprocessor for medical equipment according to clause 2, wherein
the sensor is configured to detect a velocity of the fluid that is supplied to the second conduit, and
the processor is configured to estimate the second timing, based on the velocity of the fluid detected by the sensor, a length of the first conduit, and a length of the second conduit.

Clause 4: The reprocessor for medical equipment according to clause 2, wherein
the sensor is configured to detect a flow rate of the fluid that is supplied to the second conduit, and
the processor is configured to estimate the second timing, based on the flow rate of the fluid detected by the sensor, a length and a cross-sectional area of the first conduit, and a length and a cross-sectional area of the second conduit.

Clause 5: The reprocessor for medical equipment according to clause 2, wherein
the sensor is configured to detect a pressure of the fluid that is supplied to the second conduit,
the reprocessor further comprises a memory configured to store, in advance, data indicating a relationship between the pressure of the fluid detected by the sensor and a head position of the fluid flowing through the first conduit and the second conduit, and
the processor is configured to estimate the second timing by referring to the memory, based on the pressure of the fluid detected by the sensor.

Clause 6: The reprocessor for medical equipment according to clause 1, wherein
the supplying apparatus is configured to distribute and supply the fluid at a predetermined flow rate to each of the first conduit and the second conduit, and
the processor is configured to control the supplying apparatus to:
supply the fluid at the predetermined flow rate to the first conduit; and
then supply the fluid at the predetermined flow rate continuously to the first conduit and intermittently to the second conduit.

Clause 7: The reprocessor for medical equipment according to clause 1, further comprising,
a connector configured to connect the first conduit and the first tube, wherein
the gap is formed in the connector.

Clause 8: The reprocessor for medical equipment according to clause 1, wherein the medical equipment is an endoscope.

Clause 9: The reprocessor for medical equipment according to clause 2, wherein
the processor is configured to:
estimate a third timing at which the fluid is started flowing out from the gap, based on the position of the fluid in the internal conduit;
set the second timing before the third timing; and
control the supplying apparatus to start supplying the fluid to the first conduit at the second timing.

Clause 10: The reprocessor for medical equipment according to clause 9, further comprising
an acquisition apparatus configured to acquire a signal related to conduit information of the medical equipment, wherein
the processor is configured to:
acquire the conduit information based on the signal acquired by the acquisition apparatus; and
calculate the position of the fluid in the internal conduit, based on the conduit information and the physical quantity related to the fluid.

Clause 11: An operation method of a reprocessor for endoscope,
the endoscope comprising: a first conduit communicated with a first ferrule; a second conduit communicated with a second ferrule; and a third conduit where the first conduit and the second conduit are merged, the third conduit including an opening at a distal end of an insertion portion of the endoscope, the operation method comprising:
by the reprocessor, starting, at a first timing, supplying a fluid to the second conduit; and
by the reprocessor, starting, at a second timing, supplying the fluid to the first conduit via a first tube which is connected to the first conduit with a gap, and pushing the fluid back from the first conduit toward a merging point where the first conduit, the second conduit and the third conduit are merged, the second timing being before the fluid supplied to the second conduit flows out from the gap via the merging point and the first conduit.

Clause 12: The operation method of the reprocessor for endoscope according to clause 11, wherein
the reprocessor,
based on a physical quantity detected by a sensor that detects the physical quantity related to the fluid that is supplied to the second conduit by a supplying apparatus, calculates a position of the fluid, which has been started to be supplied to the second conduit at the first timing, in the conduits of the endoscope, and
estimates the second timing based on the calculated position of the fluid.

Clause 13: The operation method of the reprocessor for endoscope according to clause 12, wherein
the sensor detects a velocity of the fluid that is supplied to the second conduit, and
the reprocessor estimates the second timing, based on the velocity of the fluid detected by the sensor, a length of the first conduit, and a length of the second conduit.

Clause 14: The operation method of the reprocessor for endoscope according to clause 12, wherein
the sensor detects a flow rate of the fluid that is supplied to the second conduit, and
the reprocessor estimates the second timing, based on the flow rate of the fluid detected by the sensor, a length and a cross-sectional area of the first conduit, and a length and a cross-sectional area of the second conduit.

Clause 15: The operation method of the reprocessor for endoscope according to clause 12, wherein
the sensor detects a pressure of the fluid that is supplied to the second conduit, and
the reprocessor estimates, based on the pressure of the fluid detected by the sensor, the second timing by referring to a memory that stores, in advance, data indicating a relationship between the pressure of the fluid detected by the sensor and a head position of the fluid flowing through the first conduit and the second conduit.

Clause 16: The operation method of the reprocessor for endoscope according to clause 11, wherein
a supplying apparatus configured to distribute and supply the fluid at a predetermined flow rate to each of the first conduit and the second conduit, and
the reprocessor controls the supplying apparatus to:
supply the fluid at the predetermined flow rate to the first conduit; and
then supply the fluid at the predetermined flow rate continuously to the first conduit and intermittently to the second conduit.

Clause 17: A non-transitory computer-readable medium that records a computer program for reprocessing an endoscope, the endoscope comprising: a first conduit communicated with a first ferrule; a second conduit communicated with a second ferrule; and a third conduit where the first conduit and the second conduit are merged, the third conduit including an opening at a distal end of an insertion portion of the endoscope,
the computer program causing a computer to perform processing of:
starting, at a first timing, supplying a fluid to the second conduit; and
starting, at a second timing, supplying the fluid to the first conduit via a first tube which is connected to the first conduit with a gap, and pushing the fluid back from the first conduit toward a merging point where the first conduit, the second conduit and the third conduit are merged, the second timing being before the fluid supplied to the second conduit flows out from the gap via the merging point and the first conduit.

Clause 18: The non-transitory computer-readable medium according to clause 17, wherein
the computer program causes the computer to perform processing of:
based on a physical quantity detected by a sensor that detects the physical quantity related to the fluid that is supplied to the second conduit by a supplying apparatus, calculating a position of the fluid, which has been started to be supplied to the second conduit at the first timing, in the conduits of the endoscope; and
estimating the second timing based on the calculated position of the fluid.

Clause 19: The non-transitory computer-readable medium according to clause 18, wherein
the computer program causes the computer to perform processing of estimating the second timing, based on a velocity of the fluid that is supplied to the second conduit, the velocity being detected by the sensor, a length of the first conduit, and a length of the second conduit.

Clause 20: The non-transitory computer-readable medium according to Clause 18, wherein
the computer program causes the computer to perform processing of estimating the second timing, based on a flow rate of the fluid that is supplied to the second conduit, the flow rate being detected by the sensor, a length and a cross-sectional area of the first conduit, and a length and a cross-sectional area of the second conduit.

What is claimed is:

1. A reprocessor comprising:
a first tube connectable to a first conduit of medical equipment with a gap therebetween;
a second tube connectable to a second conduit of the medical equipment;
a supplying apparatus configured to supply a fluid including a liquid to the first conduit via the first tube and to supply the fluid to the second conduit via the second tube; and
a processor configured to control the supplying apparatus to:
start supplying the fluid to the second conduit at a first timing; and
start supplying the fluid to the first conduit at a second timing before the fluid supplied to the second conduit flows out from the gap via a merging point and the first conduit, thereby pushing the fluid back toward the merging point from the first conduit,
wherein the medical equipment comprises an internal conduit including the first conduit, the second conduit, and a third conduit, each of the first to third conduits being open at one end and merging with the other two conduits at the merging point at another end.

2. The reprocessor according to claim 1, further comprising a sensor configured to detect a physical quantity relating to the fluid supplied from the supplying apparatus to the second conduit,
wherein the processor is configured to:
calculate a position, in the internal conduit, of the fluid having started to be supplied to the second conduit at the first timing, based on the physical quantity detected by the sensor; and
estimate the second timing based on the calculated position of the fluid.

3. The reprocessor according to claim 2, wherein
the sensor is further configured to detect a velocity of the fluid supplied to the second conduit, and
the processor is further configured to estimate the second timing based on the detected velocity of the fluid and lengths of the first conduit and the second conduit.

4. The reprocessor according to claim 2, wherein
the sensor is further configured to detect a flow rate of the fluid supplied to the second conduit, and
the processor is further configured to estimate the second timing based on the detected flow rate of the fluid, a length and a cross-sectional area of the first conduit, and a length and a cross-sectional area of the second conduit.

5. The reprocessor according to claim 2, wherein
the sensor is further configured to detect a pressure of the fluid supplied to the second conduit,
the reprocessor further comprises a memory configured to store data indicating a relationship between the detected pressure of the fluid and a leading-end position of the fluid flowing through the first conduit and the second conduit, and
the processor is further configured to estimate the second timing by referring to the memory based on the detected pressure of the fluid.

6. The reprocessor according to claim 1, wherein
the supplying apparatus is further configured to distribute and supply the fluid at a predetermined flow rate to the first conduit and the second conduit, and
the processor is further configured to control the supplying apparatus to:
supply the fluid at the predetermined flow rate to the first conduit; and
then supply the fluid at the predetermined flow rate continuously to the first conduit and intermittently to the second conduit.

7. The reprocessor according to claim 1, further comprising a connector having the gap therein and configured to connect the first tube to the first conduit.

8. The reprocessor according to claim 1, wherein the medical equipment is an endoscope.

9. The reprocessor according to claim 2,
wherein the processor is further configured to:
estimate a third timing at which the fluid starts flowing out from the gap based on the position of the fluid in the internal conduit;
set the second timing before the third timing; and
control the supplying apparatus to start supplying the fluid to the first conduit at the second timing.

10. The reprocessor according to claim 9, further comprising an acquisition apparatus configured to acquire a signal relating to conduit information of the medical equipment,
wherein the processor is further configured to:
acquire the conduit information from the signal acquired by the acquisition apparatus; and
calculate the position of the fluid in the internal conduit based on the conduit information and the physical quantity relating to the fluid.

11. A method implementable by a reprocessor comprising a first tube connectable to a first conduit of an endoscope with a gap therebetween, and a second tube connectable to a second conduit of the endoscope, the method comprising:
starting to supply a fluid to the second conduit via the second tube at a first timing; and
starting to supply the fluid to the first conduit via the first tube at a second timing before the fluid supplied to the second conduit flows out from the gap via a merging point and the first conduit, thereby pushing the fluid back toward the merging point from the first conduit,
wherein the endoscope comprises an internal conduit including the first conduit, the second conduit, a third conduit having an opening at a distal end of an insertion portion of the endoscope, the first, second, and third conduits merging with each other at the merging point.

12. The method according to claim 11, further comprising:
calculating a position, in the internal conduit, of the fluid having started to be supplied to the second conduit at the first timing, based on a physical quantity detected by a sensor, the physical quantity relating to the fluid supplied to the second conduit; and
estimating the second timing based on the calculated position of the fluid.

13. The method according to claim 12, wherein
the sensor is further configured to detect a velocity of the fluid supplied to the second conduit, and
the estimating includes estimating the second timing based on the detected velocity of the fluid and lengths of the first conduit and the second conduit.

14. The method according to claim 12, wherein
the sensor is further configured to detect a flow rate of the fluid supplied to the second conduit, and
the estimating includes estimating the second timing based on the detected flow rate of the fluid, a length and a cross-sectional area of the first conduit, and a length and a cross-sectional area of the second conduit.

15. The method according to claim 12, wherein
the sensor is further configured to detect a pressure of the fluid supplied to the second conduit, and
the estimating includes:
referring to a memory storing data indicating a relationship between the detected pressure of the fluid and a leading-end position of the fluid flowing through the first conduit and the second conduit; and
estimating the second timing based on the detected pressure of the fluid and the data stored in the memory.

16. The method according to claim 11, further comprising:
controlling a supplying apparatus to:
supply the fluid at a predetermined flow rate to the first conduit; and
then supply the fluid at the predetermined flow rate continuously to the first conduit and intermittently to the second conduit,
wherein the supplying apparatus is configured to distribute and supply the fluid at the predetermined flow rate to the first conduit and the second conduit.

17. A non-transitory computer-readable medium storing computer-readable instructions executable by a processor of a reprocessor comprising a first tube connectable to a first conduit of an endoscope with a gap therebetween, and a second tube connectable to a second conduit of the endoscope, the computer-readable instructions being configured to, when executed by the processor, cause the reprocessor to execute the method according to claim 11.

18. The non-transitory computer-readable medium according to claim 17,
wherein the computer-readable instructions are further configured to, when executed by the processor, cause the reprocessor to execute the method according to claim 12.

19. The non-transitory computer-readable medium according to claim 18, wherein
the sensor is further configured to detect a velocity of the fluid supplied to the second conduit, and
the computer-readable instructions are further configured to, when executed by the processor, cause the reprocessor to execute the method according to claim 13.

20. The non-transitory computer-readable medium according to claim 18, wherein
the sensor is further configured to detect a flow rate of the fluid supplied to the second conduit, and
the computer-readable instructions are further configured to, when executed by the processor, cause the reprocessor to execute the method according to claim 14.
